(19) United States
(12) Patent Application Publication
(10) Pub. No.: US 2012/0058197 A1
(43) Pub. Date: Mar. 8, 2012

(54) COMPOSITIONS COMPRISING WATER WITH DEUTERIUM FOR THE PREVENTION OR TREATMENT OF ALLERGIC DISEASES AND A PROCESS FOR THE PREPARATION THEREOF

(76) Inventor: Gábor Somlyai, Budapest (HU)

(21) Appl. No.: 13/264,068

(22) PCT Filed: Apr. 16, 2010
(86) PCT No.: PCT/HU10/00044

§ 371 Date: Nov. 11, 2011

(30) Foreign Application Priority Data
Apr. 16, 2009 (HU) P0900231

(51) Int. Cl. A61K 33/00; A61P 37/08

(52) U.S. Cl. 424/600

(57) ABSTRACT

The invention relates to pharmaceutical and food compositions for the prevention or treatment of allergic diseases and a process for the preparation thereof. The process of the invention comprises mixing water having 0.01-135 ppm deuterium content (preferably prepared by known electrolysis or distillation) with usual additives, and formulating this mixture by usual pharmaceutical or food industrial methods to pharmaceutical or food products, preferably by applying standard flavors, aromas and other additives used in the manufacturing of medicines, non-alcoholic drinks or beer.

The subject of the invention relates to pharmaceutical and food industrial compositions for the prevention or treatment of allergic diseases and a process for the preparation thereof.

### BACKGROUND OF THE INVENTION

In the recent years, a number of scientific publications (FEBS Lett. 317:1-4, 1993; Természetgyógyászat 10:29-32, 1996; Kisállatorvoslás 3: 114-5, 1996; Erfahrungsheilkunde 7:381-88, 1997; J. R. Heys and D. G. Melillo (eds) Synthesis and Applications of Isotopically Labelled Compounds. John Wiley and Sons Ltd. pp. 137-141, 1997) as well as Hungarian patents (Reg. No.: 208084, 209787, 214593, 214824) were based on the recognition that naturally occurring deuterium plays an important role in the regulation of cell division. Beyond the animal experiments described in the patents referred to, the antitumor effect of deuterium-depleted water (abbreviated as “Dd water” and deuterium is abbreviated as D in the following parts) has been proven also in human test experiments. In a two-year long human phase II double-blind clinical study (where neither the attending physicians nor the subjects were aware of which of the patients had received the agent and which the placebo) significant difference was observed between the treated and control group of prostate cancer patients. This study unequivocally confirmed the results of our earlier animal and human investigations.

Independently of the clinical trials, altogether 1290 patients consumed 400 tons of Dd water in the past 15 years. These cases underlined that tumour cells react sensitively on deuterium depletion and are, in the overwhelming majority of cases (70-80%), unable to adapt to lowered deuterium concentrations, resulting in partial or complete regression of the tumour mass.

A number of interesting observations were taken during the more than 15 years long period of patient follow-up. The observations on allergic patients brought the unexpected result that Dd water reduced or eliminated the complaints typical for an allergic disease (details are discussed below).

The invention is based on the observation that consuming of Dd water as drinking water prevents, diminishes or eliminates the symptoms of allergic diseases and is thus suitable for prevention or treatment of the disease.

### SUMMARY OF THE INVENTION

As a first aspect, the invention relates to a process for the preparation of pharmaceutical and food compositions comprising water having 0.01-135 ppm deuterium content, which are suitable for the prevention or treatment of allergic diseases. The applied Dd water having 0.01-135 ppm deuterium content is prepared by known electrolysis and/or distillation preferably or by other methods [Howard K. Rae (ed.) Separation of Hydrogen Isotopes, American Chemical Society Symposium Series 68, Washington D.C. 1978; Stelio Villani (ed.) Isotope Separation. American Nuclear Society 1983] and the obtained water is formulated into pharmaceutical or food composition by adding usual additives (e.g. flavours, aromas etc.) and by applying usual methods of the field, especially which are used in manufacturing of medicines, non-alcoholic drinks or beer.

As another aspect, the invention relates to

a) pharmaceutical composition for the prevention or treatment of allergic diseases, which comprises water having 0.01 to 135 ppm deuterium content together with usual additives applied in the pharmaceutical industry; and

b) food composition for the prevention or treatment of allergic diseases, which comprises water having 0.01 to 135 ppm deuterium content together with usual additives applied in the food industry.

In both of the above composition the water having 0.01 to 135 ppm deuterium content is prepared by known electrolysis and/or distillation or any other known methods resulting in Dd water.

In a preferred embodiment the applied Dd water has a deuterium concentration of 5 to 125 ppm, more preferably 50 to 110, and further more preferably 75 to 90 ppm. However, for human consumption, especially in the first 2-3 months of the treatment, the preferred Dd content is 105 to 130 ppm and in the next 2-3 months 80-105 ppm.

It should be noted that if the Dd water administration was not successful as prevention, then Dd water with a less D-concentration can be applied in the treatment phase. Moreover, after a successful treatment with Dd water, it can be applied as preventive agent against a future allergic attack, with the same or different D-concentration. So, in the case of Dd water the prevention and treatment can be applied consequently, completing each other.

### DETAILED DESCRIPTION OF THE INVENTION

The deuterium concentration of water was determined by infrared spectrometry, based on the absorption maximum of the O-D bond at 4 μm wavelength. Filling the water sample in the cuvette, infrared light passing through the water will be absorbed in proportion to the deuterium content, what is detected by the instrument. After calibration of the instrument with water of known D content, the amount of D in the test samples can be determined (Analytical Biochemistry 98:208-213, 1979). Below we describe two preferred processes for the production of Dd water, i.e. electrolysis and distillation, which are suitable for making Dd-water in large quantity at a relatively low cost.

a) An aqueous KOH solution of 15-20% is electrolysed by direct current at a potential of 2-5 V with a cathode and an anode separated from each other. The hydrogen evolving on the cathode and containing deuterium in a decreased concentration is burnt and the steam being formed is condensed and separately collected. The obtained water has a deuterium content of 30-40 ppm (Separation of Hydrogen Isotopes Eds.: Howard K. Rae, American Chemical Society Symposium Series 68, Washington D.C. 1978; Isotope Separation Eds.: Stelio Villani, American Nuclear Society 1983). The deuterium content of the obtained water can be further decreased 6 to 20 ppm by a further electrolysis.

b) The water is distilled in a fractionating column of 50 to 150 plates under pressure of 50 to 60 mbar and at a temperature of 45 to 50° C. The reflux value is 12 to 13 in the course of the distillation and a tenfold dilution at the bottom is applied during the distillation. Applying these parameters, the deuterium concentration of the head-products is between 0.1 and 30 ppm (Separation of Hydrogen Isotopes Eds.: Howard K. Rae, American Chemical Society Symposium Series 68, Washington D.C. 1978; Isotope Separation Eds.: Stelio Villani, American Nuclear Society 1983). Through changing the parameters in the course of operation of the column, e.g. through significantly increasing the load of the column, Dd-water with higher D concentration than 30 ppm can be produced in large quantities. It is a further possibility to decrease the D-concentration of the water to subject Dd-water to further fractionating distillation process on a similar, joint column.

Dd water, produced by the above described (or by any other) methods is used as starting raw material.

The phrase “pharmaceutical composition” as it is used in this specification includes the veterinary compositions, too.

As for the phrase “allergic diseases” we would give the following interpretation:

Allergies occur when the immune system reacts against harmless substances in the environment. The immune system is extremely complex and so there are many opportunities for things to go wrong. When the immune system begins to react against harmless substances in the environment, this can lead to allergic reactions, which are exaggerated, damaging immune responses to substances that are normally harmless.

When people with allergy diseases are exposed to common environmental substances such as house dust mite or grass pollens, a type of white blood cell (B lymphocytes) produce specific antibodies known as IgE against that substance. This IgE then attaches itself to another type of white blood cell (mast cells), and when the mast cells come into contact with that substance again, they initiate a complex immune response that leads to the allergy.

Different people with allergies are allergic to different substances. Some of the substances that people are commonly allergic to include: house dust mite, pollens from grasses and trees, animal dander including cat, dog, horse, moulds, foods including tree nuts, peanuts, shellfish, fish, milk, eggs, wheat and more.

Different people with allergies also react differently when they are exposed to the substance they are allergic to. Some common reactions include: allergic eczema or urticaria, allergic rhinitis (hay fever), allergic asthma, anaphylaxsis, sneezing, nasal congestion and eye irritation. Similar symptoms occur in some animals, too.

During the formulation of the pharmaceutical or food compositions according to the invention, any usual additives [e.g. sweeteners, colorants, thickeners, flavours (flavouring agents), pH-adjusters, surfactants, aromas, fruit concentrates, stabilizers etc.] can be applied which are known in the pharmaceutical industry (including the industry producing veterinary compositions, to) and food industry, in the latter case especially those which are applied in manufacturing of beer and non-alcoholic drinks. Here we would emphasize that the additive can be water having normal (natural) deuterium content.

During the preparation of the compositions according to the invention, any standard (known) formulation method of pharmaceutical and food industry (including the industry producing veterinary compositions, too) can be applied.

It comes from the use of Dd water in the preparation process of the invented compositions that the liquid form is an advantageous formulation type. However, especially in the food industrial application, the prepared composition can be in solid (or semi-solid, gel, colloidal mixture etc.) form. In this case the formulation contains only Dd water, i.e. by the consumption of it the average deuterium content of the treated body is not increased, i.e. these formulation can supplement the basic cure made by the liquid form composition.

The main advantages of the procedure of the invention are as follows:

a) It allows intervention without synthetic “medicines” into allergic processes and beneficially influences the course of disease,

b/ Dd water used in the procedure has no toxic side effects.

c/ The production process generates no hazardous waste.

d/ The compositions are manufactured easily.

As it was mentioned above, some important positive effect of Dd water in the treatment of allergic diseases were found in our practice. The most valuable results are summarized as follows:

One of the groups of allergy patients (10 patients) suffered from hay fever every year mainly in spring time. They reported to our team that after the consumption of Dd water with a D-content of 105 or 125 ppm, the symptoms completely disappeared or significantly weakened.

Another patient suffered every year from stronger symptoms of sneezing, nasal congestion and eye irritation, but after starting the consumption of Dd water (85 ppm) 3-4 weeks before the regular appearance of the symptoms, they all could have been prevented.

One of the patient with severe allergic reaction took medication for long period of time to attenuate the symptoms. After she had started to consume Dd water (85 ppm) she could quit taking drugs.

Concerning the possibility of veterinary use of Dd water, we can report a positive effect of Dd water on a horse suffering from very serious nasal congestion. After the horse was consuming apprx. 150-200 litres of Dd water with 85 ppm D-content it became asymptomatic. The most surprising result was that after this one and only Dd water cure, the symptoms did not appear in the forthcoming years.

As it comes from the above practical experiences, the composition obtained by the process according to the invention is suitable for the prevention or treatment of allergic diseases. The molecular mechanisms which might be crucial in this effect are being studied. Preliminary results suggest that the decrease of D level in the organism, induced by Dd water, might influence the expression of certain genes. An explanation of the improvement or elimination of symptoms might be that the alterations of gene expression induced by the Dd water adequately respond to the causes triggering the allergic symptoms. The results obtained with Dd water in humans testify the suitability of the process according to the invention for treatment of allergic diseases.

Albeit detailed statistically supported pharmacological studies have not been carried out yet, the following considerations can be made to discover the theoretical background of the pharmaceutical effect found. However, we do not bind ourselves to the theories discussed below with respect to complicated and mainly undiscovered theoretical background of allergy.

Pharmacological Studies and Experiments

An allergic response is a hypersensitive immune reaction to a substance that normally is harmless or would not cause an immune response in everyone.

Lymphocytes are white blood cells that play a key role in both immunity and allergy. They are divided into two types, the T and B lymphocytes. Each type is responsible for a particular branch of the immune system. It is the duty of the T-lymphocytes to be ready to directly shift into action to attack foreign substances (cell-mediated immunity). Some T-lymphocytes are experts at “killing” (cytotoxic or killer T cells) while others assist the immune response and are termed “helper” cells (TH cells). The TH cells are further divided into TH1 (infection fighters) and TH2 (allergy promoters), depending on the proteins they release. The partners of the T-lymphocytes are the B-lymphocytes. B-lymphocytes are tiny antibody factories that produce antibodies to help destroy foreign substances when stimulated to do so by the TH cells.

Basophils and eosinophils are other white blood cells that play an important role in allergy. T cells often call these cells into action in allergic conditions. Blood levels of eosinophils are commonly elevated in people with asthma and other allergic diseases.

Cytokines are small proteins that are released by lymphocytes can either step-up or step-down the immune response. One of the cytokines, interleukin 4 (IL4), is essential for the production of IgE. Interleukin 5 (IL5) and others are important in attracting other cells, particularly eosinophils, which then promote inflammation. This spectrum of cytokines is also released by the TH2 lymphocytes, thus further promoting allergic inflammation.

Three main points of therapeutic intervention are proposed for the treatment of allergic disease. A) The first possibility is to block initiation of the immune response and thereby prevent development of disease-promoting T helper 2 (T_{H}2) responses to allergens: for example, by intervention early in life. B) The second possibility is to block activation of allergen-specific T_{H}2 cells, either directly or indirectly through effects on antigen-presenting cells: for example, by treatment with anti-inflammatory drugs, such as glucocorticoids, or by allergen immunotherapy. C) The third possibility is to block effector molecules that cause the clinical symptoms of allergic disease: for example, by treatment with antihistamines, leukotriene antagonists, neutralizing antibodies specific for T_{H}2 cytokines or antibodies specific for IgE. IL, interleukin; TCR, T-cell receptor (Nature Reviews Immunology 5, 271-283 (April 2005) Potential role of interleukin-10-secreting regulatory T cells in allergy and asthma C. M. Hawrylowicz & A. O'Garra).

Other results revealed that tyrosine phosphorylation is critical to IL-2-mediated signal transduction and that MAP kinase is one of the cellular intermediates involved in this pathway. (Activation of mitogen-activated protein kinase/ERK-2 in phytohaemagglutin in blasts by recombinant interleukin-2: contrasting features with CD3 activation. R M Fairhurst, M Daeipour, M C Amaral, and A E Nel, Immunology. 1993 May; 79(1): 112-118.) An other paper also proves the involvement of MAP kinase in IL-induced chemokine release (Involvement of p38 MAPK, JNK, p42/p44 ERK and NF-κB in IL-1β-induced chemokine release in human airway smooth muscle cells (Wim A. Wuyts, Bart M. Vanaudenaerde, Lieven J. Dupont, Maurits G. Demedts and Geert M. Verleden. Respiratory Medicine 2003 July, 97(7): 811-817).

To reveal the specific pathways what the application of deuterium depletion use to attenuate or prevent the allergic reaction needs further basic and clinical research, but based upon the results available there are clear evidences that Dd water influences the molecular mechanism having key role in allergic reaction.

As it was mentioned above that blocking of activation of allergen-specific T_{H}2 cells, either directly or indirectly through effects on antigen-presenting cells—for example, by treatment with anti-inflammatory drugs—is one possibility to treat allergic disease.

It is well known that the main target of non-steroidal anti inflammatory drug is the COX-2 gene responsible for prostaglandin synthesis. Experiments were carried out with a tumorous cell line of human colon origin (adenocarcinoma). The cells were cultured in RPMI media containing 10 percent fetal calf serum (FCS) in a carbon-dioxide thermostat (5 percent CO₂:95 percent air). When cells had almost filled the space in the culture dish (70 percent confluence value), the serum was withdrew for 24 hours. Following serum deprivation, the cells were given RPMI media prepared with deuterium depleted water, and after 24 hours the MTT (microculture tetrazolinum) assay was performed to determine cell proliferation. With Western blot analysis we measured the COX-2 content of cells treated as detailed above. Assay has shown that Dd-water (20-60 ppm) strongly inhibits the COX-2 gene expression of HT-29 human colon carcinoma cell lines. Similar to the results of cell proliferation experiments, here too, the effect proved to depend on the concentration. FIG. 1. shows the effect of D₂O concentration on COX-2 gene expression. FIG. 2. shows the correlation between the D-concentration and the amount of intracellular prostaglandin confirming that the inhibitory effect of Dd water on COX-2 gene expression resulted a lower concentration of prostaglandin in the cells (Gábor Somlyai: Let's defeat cancer. Published by Akadémiai Kiadó, P.O. Box 245. H-1519 Budapest, www.akkrt.hu, ISBN 963 05 7807 6).

These results may suggest that one of the mode of action of Dd water on allergic patients can be the inhibitory effect of COX-2 gene. However, it should be understood that from the COX-2 gene inhibitory effect does not come the antiallergic effect. This phenomenon reveals such an effect which can be one of those which play a role in the treatment of allergy.

It was also mentioned above that the MAP kinase (ERK) phosphorylation is critical to IL-2-mediated signal transduction. FIG. 3. shows (unpublished results) that in normal water (150 ppm) the phosphorilated band appeared 2 minutes after the induction but in Dd water (20 ppm) the phosphorilation did not occur. This clearly suggest that the application of Dd water intervene into the signal transduction pathways and may inhibit the IL-mediated signal transduction. However, it should be understood that from the phosphorylation inhibitory effect does not come the antiallergic effect. This phenomenon reveals such an effect which can be one of those which play a role in the treatment of allergy.

As it was discussed above, the signal transduction system exerts an effect on allergic reaction, but the way of it is very complicated and mainly undiscovered. As it comes from the results obtained in the study of the effect of D-concentration on the modification of the levels of different ILs (Table 1; the experiments were made in a competent independent laboratory), it can be said that the Dd water has a complicated effect on the IL levels. However, the found positive effect on the treatment of allergic diseases cannot be predicted on the basis of the obtained experimental results (mainly decreased but sometimes increased levels were found for the different ILs). Only one fact can be deducted from the results: Dd water has an influence on the IL levels which play an important role in allergy.

Tables 1 to 3 below summaries the effects of Dd water on cytokine concentrations (levels). Values significantly higher than the Sterile Water Control (p≦0.05) are shown in bold. Values significantly lower than the Sterile Water Control (p≦0.05) are shown in bold and italic. It should be noted that levels of IL-8, IL-6, MCP-1, and G-CSF were too high in all groups to be quantified in the present dilution scheme.

TABLE 1
(DDW: Dd water)

| | | | IL1b, | | IL2, | |
|---|---|---|---|---|---|---|
| | IL1a, % of | | % of | | % of | |
| | Sterile | Std. | Sterile | Std. | Sterile | Std. |
| | Water | Dev. | Water | Dev. | Water | Dev. |
| Sterile Water (~150 | 100.00 | 72.16 | 100.00 | 43.41 | 100.00 | 53.98 |
| ppm) | | | | | | |
| DdW, 150 ppm | 115.84 | 10.32 | 115.32 | 63.39 | 117.96 | 33.05 |
| DdW, 130 ppm | 85.79 | 27.12 | 82.45 | 30.92 | 115.74 | 29.87 |
| DdW, 110 ppm | 68.76 | 20.52 | 91.64 | 44.89 | 117.52 | 54.81 |
| DdW, 80 ppm | 168.44 | 43.72 | 102.23 | 36.92 | 98.23 | 28.61 |
| DdW, 0.5 ppm | [symbol] | 18.03 | 100.56 | 42.50 | 84.92 | 41.51 |

TABLE 2
(DDW: Dd water)

| | IL4, | | IL10, | | IL-12, | |
|---|---|---|---|---|---|---|
| | % of | | % of | | % | |
| | Sterile | Std. | Sterile | Std. | of Sterile | Std. |
| | Water | Dev. | Water | Dev. | Water | Dev. |
| Sterile Water (~150 | 100.00 | 48.63 | 100.00 | 16.90 | 100.00 | 22.54 |
| ppm) | | | | | | |
| DdW, 150 ppm | 143.38 | 29.18 | 94.76 | 29.96 | 98.51 | 31.02 |
| DdW, 130 ppm | 134.98 | 32.14 | 69.00 | 14.95 | 110.45 | 10.34 |
| DdW, 110 ppm | 121.35 | 31.96 | 63.55 | 24.20 | 88.06 | 37.24 |
| DdW, 80 ppm | 183.81 | 18.35 | 124.71 | 38.70 | 91.04 | 32.10 |
| DdW, 0.5 ppm | 107.25 | 57.18 | [symbol] | 5.52 | 89.55 | 32.70 |

TABLE 3
(DDW: Dd water)

| | | | IL-15, | | IL-17, | |
|---|---|---|---|---|---|---|
| | IL-13, % | | % of | | % of | |
| | of Sterile | Std. | Sterile | Std. | Sterile | Std. |
| | Water | Dev. | Water | Dev. | Water | Dev. |
| Sterile Water (~150 | 100.00 | 33.57 | 100.00 | 10.86 | 100.00 | 20.90 |
| ppm) | | | | | | |
| DdW, 150 ppm | 114.11 | 43.25 | 107.33 | 18.98 | 89.29 | 19.04 |
| DdW, 130 ppm | 109.93 | 23.54 | 83.42 | 15.57 | 95.41 | 35.41 |
| DdW, 110 ppm | 139.80 | 84.00 | 80.27 | 15.09 | 105.87 | 48.00 |
| DdW, 80 ppm | 128.03 | 69.27 | 124.73 | 8.22 | 63.27 | 17.42 |
| DdW, 0.5 ppm | 137.19 | 99.41 | [symbol] | [symbol] | 90.05 | 54.07 |

The effect of deuterium depleted water also was investigated in full-thickness skin equivalent cultures. After 24 hours of treatment, deuterium-depleted water had no effect on culture viability, procollagen I c-peptide production or hyaluronic acid production, so no toxic effect was found.

### SUMMARY OF THE FIGURES

FIG. 1 shows the effect of D₂O on COX-2 expression in HT %-29 cells at different concentrations.

FIG. 2 shows the effect of DDW on the prostaglandin production in Hat different concentrations.

FIG. 3 shows the effect of D₂O on FCS-induced phosphorilation of MAP kinase (ERK) in HT-29 cells.

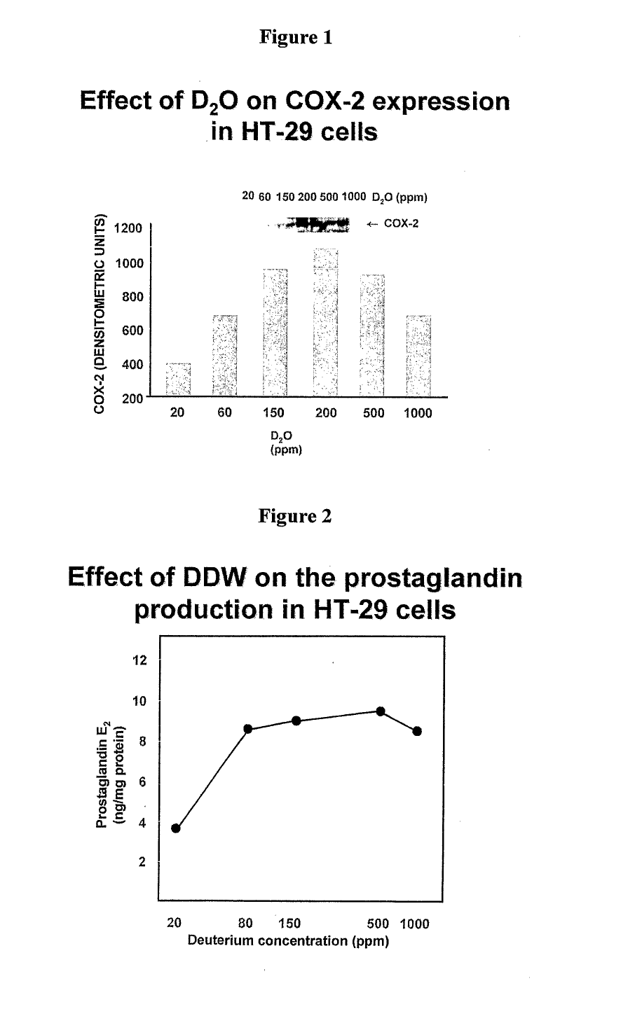

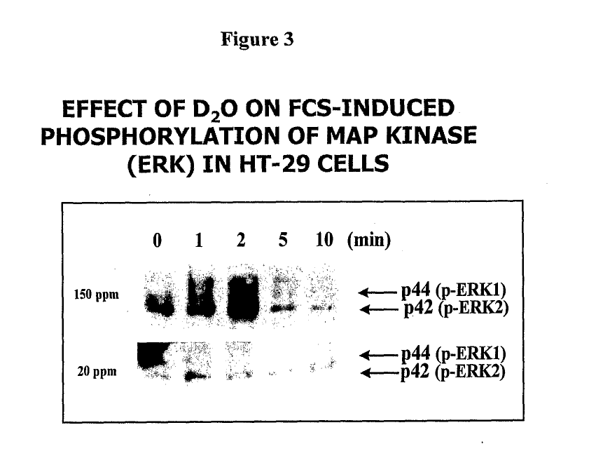

### EXAMPLES

The process according to the invention is demonstrated, without limiting the scope of protection, by the following examples:

### Example 1

### Production of Drinking Water with Advantageous Mineral Composition

Dd water and a natural mineral water with known salt composition (such as the Hungarian sorts Csillaghegyi, Balfi, Óbudai gyémánt) are mixed at ratios as follows:
a) 0.25 parts by volume of water with 90 ppm D content+0.75 parts by volume of mineral water; final D concentration 135 ppm.
b) 0.5 parts by volume of water with 90 ppm D content+0.5 parts by volume of mineral water; final D concentration 120 ppm.
c) 0.75 parts by volume of water with 90 ppm D content+0.25 parts by volume of mineral water; final D concentration 105 ppm.
d) 0.25 parts by volume of water with 60 ppm D content+0.75 parts by volume of mineral water; final D concentration 127.5 ppm.
e) 0.5 parts by volume of water with 60 ppm D content+0.5 parts by volume of mineral water; final D concentration 105 ppm.
f) 0.75 parts by volume of water with 60 ppm D content+0.25 parts by volume of mineral water; final D concentration 82.5 ppm.

### Example 2

The cation and anion content of the Dd water is set by an artificial concentrate of advantageous salt composition. This stock solution might have the composition as follows:

| | |
|---|---|
| KCl | 5.7 g |
| MgCl₂•6H₂O | 199.65 g |
| CaCl₂•6H₂O | 236.25 g |
| + Dd water to 1000 ml. | |

The stock solution made up this way yields the following final concentrations: Mg²⁺: 23.8 mg/L; Ca²⁺: 64.1 mg/L; K⁺: 3 mg/L; Cl⁻: 192 mg/L.

### Example 3

Production of Fruit Drinks and Carbonated Drinks with Reduced D Content.

Distilled water of 30 ppm D content is mixed with water or fruit juice concentrate at the following ratios:
a) 0.13 parts by volume of water with 30 ppm D content+0.67 parts by volume of water+0.20 parts by volume of fruit juice concentrate; final D concentration ca. 128-130 ppm.
b) 0.20 parts by volume of water with 30 ppm D content+0.60 parts by volume of water+0.20 parts by volume of fruit juice concentrate; final D concentration ca. 118-120 ppm.
c) 0.24 parts by volume of water with 30 ppm D content+0.56 parts by volume of water+0.20 parts by volume of fruit juice concentrate; final D concentration ca. 111-113 ppm.

### Example 4

### Production of Deuterium-Depleted Beer

The barley used for producing the malt for beer brewing is first soaked in Dd water (D concentration: 0.01 to 135 ppm), then spread in a 5 to 15 cm thick layer and malted at good aeration at low temperature (5 to 15° C.). The long malt is kiln-dried between 56 and 75° C., then the residues of germ roots are removed and the malt is ground. The ground malt is mixed to an appropriate amount of Dd water (D level: 0.01 to 135 ppm), kept at 50 to 75° C., then filtered and cooked with hops. The hoped beer-wort is filtered, cooled, then inoculated with pre-grown Saccharomyces cerevisiae. Main fermentation lasts 10 to 14 days at 5 to 6° C. The final fermentation is done in air-tight casks at 0° C. for several weeks. The beer is then filtered, bottled and pasteurized. The deuterium content of the beer produced is determined primarily by the D content of the water used, influencing also the D content of ethanol and other components.

1. Process for the preparation of a pharmaceutical composition for the prevention or treatment of allergic diseases, which comprises mixing water having 0.01 to 135 ppm deuterium content with usual additives applied in the pharmaceutical industry and formulating the obtained mixture into a pharmaceutical composition.

2. Process for the preparation of a food composition for the prevention or treatment of allergic diseases, which comprises mixing water having 0.01 to 135 ppm deuterium content with usual additives applied in the food industry and formulating the obtained mixture into a food composition.

3. The process according to claim 1, where the water having 0.01 to 135 ppm deuterium content is prepared by electrolysis or distillation.

4. The process according to claim 1, where the deuterium content is 80 to 130 ppm.

5. A pharmaceutical or food composition for the prevention or treatment of allergic diseases, which comprises water having 0.01 to 135 ppm deuterium content together with usual additives applied in the pharmaceutical or food industry.

6. The composition of claim 5 which is a food composition which comprises water having 0.01 to 135 ppm deuterium content together with usual additives applied in the food industry.

7. Composition according to claim 5, where the water having 0.01 to 135 ppm deuterium content is prepared by electrolysis or distillation.

8. The composition according to claim 5, where the deuterium content is 80 to 130 ppm.

9. The process according to claim 2, where the water having 0.01 to 135 ppm deuterium content is prepared by electrolysis or distillation.

10. The process according to claim 2, where the deuterium content is 80 to 130 ppm.

11. A method for the prevention or treatment of an allergic disease, which method comprises administering the composition of claim 5 to a patient suffering from the disease.
